(19) United States
(12) Patent Application Publication
(10) Pub. No.: US 2025/0352162 A1
(43) Pub. Date: Nov. 20, 2025

(54) REGULATING APPARATUS FOR DOSE LIMITATION OF RADIATION SOURCES

(71) Applicant: Siemens Healthineers AG, Forchheim (DE)

(72) Inventors: Thomas WEBER, Hausen (DE); Peter Horst SCHEUERING, Fuerth (DE); Maximilian LEY, Cadolzburg (DE)

(73) Assignee: Siemens Healthineers AG, Forchheim (DE)

(21) Appl. No.: 19/206,202

(22) Filed: May 13, 2025

(30) Foreign Application Priority Data
May 14, 2024 (DE) 10 2024 204 439.3

(51) Int. Cl. A61B 6/00; A61B 6/42

(52) U.S. Cl. CPC A61B 6/542; A61B 6/4208; A61B 6/548

(57) ABSTRACT

A regulating apparatus for dose limitation of radiation sources configured to generate radiation via a radiation generator, the regulating apparatus comprising: a radiation sensor configured to measure an intensity of radiation from the radiation source; and a control unit configured to regulate an intensity of the radiation emitted from the radiation source. The regulating apparatus is configured to regulate the radiation from the radiation source based on a measurement of the radiation sensor within a period of time shorter than 2 ms after the measurement.

### CROSS-REFERENCE TO RELATED APPLICATION(S)

The present application claims priority under 35 U.S.C. §119 to German Patent Application No. 10 2024 204 439.3, filed May 14, 2024, the entire contents of which are incorporated herein by reference.

### FIELD

One or more example embodiments of the present invention relate to a regulating apparatus for dose limitation of radiation sources, a medical technology system for imaging and/or radiotherapy and a method for dose limitation of a radiation source of a medical technology system.

### BACKGROUND

In a medical application of high-energy radiation, for example, X-rays, electron radiation or proton radiation, a patient must not be exposed to an excessive dose. In general, therefore, it must be ensured by standards, for example in radiography, that a (regionally differently defined) maximum dose rate, for example, an air kerma rate, or a dose, for example, an air kerma, is not exceeded at a point of reference. An explicit air kerma limit does not currently exist due to technical limitations. Added to this, the dose yield varies greatly depending on the use and age of the radiation source, so that the applied dose cannot be simply deduced from the current of a radiation generator.

In X-ray systems, a dose measurement chamber (AEC chamber, AEC: “Automatic exposure control”) after the patient currently determines the detector input dose and this signal is used to switch off the X-rays. This method is very prone to error due to technical limitations and also due to the human factor as the positioning of the patient has to be very precise. Further, the sensitivity of the AEC chamber does not correspond to that of the CsI flat-panel detector, thus requiring the calibration of both measuring systems to each other.

Currently, a picture-perfect real-time measurement of the dose or dose rate is not possible for technical reasons. To comply with the standard, a slow DAP chamber (DAP: “Dose Area Product”) is used, which only transmits a value with a small sampling (approx. every 5 ms). Due to the varying telegram runtimes in the system bus of a further 5 to 50 ms, in order to be able to determine a reliable value for a dose rate, several measuring points must be adjusted or averaged.

### SUMMARY

It is an object of one or more embodiments of the present invention to specify a regulating apparatus for dose limitation of radiation sources, a medical technology system for imaging and/or radiotherapy and a method for dose limitation of a radiation source of a medical technology system with which the disadvantages described above are avoided and the control of radiation in real time is enabled.

At least this object is achieved by a regulating apparatus, a medical technology system and a method as claimed.

A regulating apparatus according to embodiments of the present invention is used for dose limitation of radiation sources which generate radiation via a radiation generator. It comprises the following components:
a radiation sensor designed to measure the intensity of radiation from the radiation source,
a control unit designed to control the intensity of the radiation emitted from the radiation source, the regulating apparatus being designed to control the radiation from a radiation source based on a measurement of the radiation sensor within a defined period of time of less than 2 ms after this measurement.

First of all, it should be said that the term “dose limitation” refers to a limitation of a dose and/or a dose rate. A “dose rate” is a selective value and corresponds physically to a power (W=J/s). An air kerma rate is often used to specify a dose rate. A “dose” is a value summarized over a period of time and physically corresponds to an energy (J=Ws). An air kerma is often used to specify a dose. The dose is an integrated dose rate or a sum of measured values of the dose rate.

It can be clearly imagined that an X-ray beam must not be too intensive (a specific maximum dose rate must not be exceeded) as well as that the “total dose”, that is to say, the dose over an examination, must not be too great. In the case of a pulsed beam, care should also be taken to ensure that the “pulse dose”, i.e. the dose from a single pulse, does not exceed a maximum value.

The regulating apparatus according to embodiments of the present invention is preferably used for dose limitation of X-ray sources, but the present invention can also be advantageous for particle radiation sources. In particular, it is advantageous for controlling radiation sources which generate radiation for medical purposes or for the examination of sensitive objects via a radiation generator. This applies both to imaging and to therapy.

The regulating apparatus comprises a radiation sensor which is designed to measure a radiation intensity of the radiation source. This can be, for example, an X-ray sensor with a scintillator and a photodetector. Radiation sensors are well known in the prior art. Instead of only one sensor, for example, a diode-scintillator combination, several different radiation sensors can be used to effectively measure the beam, in particular its spectrum. Preferably, however, different pre-filter materials can also be applied via several identical radiation sensors.

Furthermore, the regulating apparatus comprises a control unit which is designed to control the intensity of the radiation emitted by the radiation source. This can be a unit which outputs a control signal which can be processed by a control facility (also referred to as a control device or controller) of the relevant radiation source, but this can also be a unit which controls the radiation source itself. It is also conceivable that the control unit is designed to move a radiopaque shutter in front of the X-ray source or to use grid pulsing. A control signal can be a signal which indicates that the radiation intensity must be reduced, it can also simply be a switch-off signal in a simple exemplary embodiment.

Basically, the control unit must only be able to output a signal which can be used to reduce or switch off a beam. Corresponding control units are known in the prior art.

An important requirement for the regulating apparatus is that it is able to control the radiation from a radiation source in a defined period of time shorter than 2 ms. To this end, its control unit must control the radiation source based on a measurement of the radiation sensor and this control must take place faster than 2 ms after this measurement. With regard to pulsed radiation, on reaching a maximum dose, embodiments of the present invention should be able to prevent the transmission of the next pulse, which would exceed the total dose.

The regulating apparatus is therefore preferably designed for control in real time. The term “real time” means, within the meaning of embodiments of the present invention, that a control takes place with a defined latency of less than 2 ms, preferably less than 1 ms, in particular less than 0.8 ms. The term “latency” means the period of time between measurement and control. The term “defined” means, within the meaning of embodiments of the present invention, that the latency is also maintained. Although control is permitted in a period of time shorter than the maximum latency, no period of time longer than the latency is permitted. This must be achieved by technical means and/or mechanisms. For example, when measuring and processing signals in the range of nanoseconds or a few microseconds, it is easy to fall short of a maximum latency of 2 ms, but the forwarding channel for control should not be overlooked either. For example, the regulating apparatus should not be designed for a signal line via Bluetooth or WLAN if it is not possible to ensure real-time communication via this channel.

The requirement for control in a defined period of time shorter than 2 ms relates to technical measures concerning a selection of components and their combination. The feature “the regulating apparatus being designed to control the radiation from a radiation source based on a measurement of the radiation sensor within a defined period of time of less than 2 ms after this measurement” is therefore synonymous with the fact that the radiation sensor and the control unit are designed and combined in such a way, and the control channel of the regulating apparatus is also designed in such a way, that the predetermined time is maintained.

On the one hand, this applies to the measurement time TM of the radiation sensor. This must be designed in such a way that its measurement time TM is very short, preferably less than 0.01 ms, in particular less than 0.001 ms, particularly preferably less than 500 ns. Customary semiconductor sensors can easily achieve this as they measure in the range of nanoseconds.

Furthermore, this applies to the transition time TU required by measured values from the radiation sensor to the control unit. A wired route should be selected, but a fast wireless channel is also possible. In addition, there may also be time added to TU for the digitization of the measured values. Customary analog-digital converters have conversion times of less than 0.01 ms. The transmission channel from the radiation sensor to the control unit should be selected in such a way that the transmission time is less than 0.1 ms.

This can be achieved using wired as well as wireless methods, but at times not with non-real-time capable methods such as Bluetooth or WLAN. However, a normal radio connection can achieve these times.

The same applies to signal transmission, the output time TA of the control unit. This too should be less than 0.1 ms. A wired output or a wireless output is possible but should not take place with non-real-time capable methods such as Bluetooth or WLAN. However, a normal radio connection can achieve these times.

As far as the control unit is concerned, this must perform arithmetic operations. The control time TR which is required to generate a control signal from the measurement signal or from several measurement signals, should be less than 1.5 ms, preferably less than 1 ms. Microcontrollers or FPGAs with a fixed workflow are very well suited. Customary processors can also be used if real-time capable processing is ensured.

The above period of time T (the latency, less than 2 ms) is the sum of these times (T=TM+TU+TR+TA). The individual components should therefore be selected according to these times or be connected to one another via data technology.

A medical technology system according to embodiments of the present invention is used in particular for imaging and/or radiotherapy. It comprises the following components:
a radiation source with a radiation generator, and
a regulating apparatus according to embodiments of the present invention, designed to control the intensity of the radiation emitted by the radiation source, the regulating apparatus being designed to initiate a switch-off of the radiation source based on the measurement of the radiation sensor if a measurement is outside a predefined range of values.

It is preferable that the radiation source is designed for a pulsed beam operation and the regulating apparatus is designed to regulate the radiation source in such a manner that on reaching a maximum dose, the radiation source is switched off, in particular before emitting the next pulse.

A medical technology system can be, for example, a radiography system, a CT system, a mammography system, a fluoroscopy system or an angiography system. However, it can also be a particle accelerator for therapeutic purposes. The system must include a radiation source with a radiation generator. Such systems are well known.

The special feature of the system is that it comprises a regulating apparatus according to embodiments of the present invention which is designed to regulate the intensity of the radiation emitted by the radiation source. This can be regulation of the radiation generator, for example, regulation of its energy supply. However, this can also include regulation of other components of the radiation source, for example, regulation of radiation filters, collimators, apertures or beam shutters. In the case of particle accelerators, regulation of magnetic fields is also possible.

The regulating apparatus is designed, based on the measurement of the radiation sensor, to initiate a reduction in the beam intensity or a switch-off of the beam of the radiation source if a measurement is outside a predefined range of values. This can be achieved, for example, by a reduction in the beam intensity, for example, by reducing the tube current, switching off the radiation generator or interrupting the beam.

Preferably, the radiation source is designed for pulsed beam operation and the regulating apparatus is designed to regulate the radiation source in such a manner that on reaching a maximum dose, the radiation source is switched off, in particular before emitting the next pulse.

In an exemplary case, there is a total limit value for a total dose for the examination, but also a pulse-limit value for a maximum pulse dose per radiation pulse and a power limit value for a maximum radiation power. These limit values are designed in such a way that they can be applied directly to measured values of the radiation sensor. Of course, it is also possible to convert each measured value of the radiation sensor into a dose rate, for example, an air kerma rate. In this case, however, the limit values were converted to the measured values based on the respective maximum value for the dose or the dose rate in order to save computing effort.

The radiation sensor now measures individual values (power values) at different times and on several occasions per pulse and for all pulses. The measured values within a pulse are added up to form a “pulse value” and all the values are added up to form a “total value”. The measured value in each case is now compared with the power limit value and the beam is switched off or its performance is reduced if the measured value is above the power limit value. Furthermore, the pulse value is compared with the pulse limit value and the beam for the current pulse is switched off if the pulse value is above the pulse limit value. Likewise, the total value is compared with the total limit value and the beam for the current examination is switched off if the total value is above the total limit value. It should be noted that the switch-off takes place in real time, i.e. at least faster than 2 ms after the measurement. This enables very fast switch-off (or control).

A method according to embodiments of the present invention is used for dose limitation of a radiation source of a medical technology system according to embodiments of the present invention for an examination. It comprises the following steps:
measurement of the intensity of the radiation of the radiation source with the radiation sensor of the regulating apparatus of the medical technology system, measurements for different radiation pulses preferably being performed via the radiation sensor,
comparison of the measured intensities and/or an integral or a sum of measured intensities with a number of predefined limit values,
if a predefined limit value is exceeded: regulation of the intensity of the radiation emitted from the radiation source by the control unit of the regulating apparatus of the medical technology system, signals from the control unit preferably being transmitted directly (within the permissible latency) to the radiation source.

It is preferable to determine whether
a measurement exceeds a predefined limit value for a dose rate, and/or
an integral or a sum of values of several measurements exceeds a predefined limit value for a total dose, and/or
an integral or a sum of values of several measurements over a radiation pulse exceeds a predefined limit value for a pulse dose.

As indicated in the example above, the intensity of the radiation from the radiation source is measured using the radiation sensor of the regulating apparatus of the medical technology system. This preferably takes place at different times on many occasions. In pulsed beam operation, several measurements should be taken within one pulse. Measurements (preferably several) are therefore preferably carried out for different radiation pulses via the radiation sensor.

The measured intensities (each corresponds to one dose rate) and/or an integral or a sum of measured intensities (corresponds to one dose) are then compared with a number of predefined limit values. The measured values can be converted to a dose or dose rate or limit values from a dose or dose rate can be adjusted to the measured values.

If a predefined limit value is exceeded, the beam intensity is then regulated, i.e. reduced or the beam is switched off. This is done by the control unit of the regulating apparatus. This preferably sends signals directly to the radiation source, for example, in the form of switch-off commands or as a numerical value for a control facility.

In this context, it is preferably determined whether a measurement (corresponds to a power value) exceeds a predefined limit value for a dose rate. In addition or alternatively, it is preferably determined whether an integral (over a period of time) or a sum of measured values of several measurements (corresponds to a dose) exceeds a predefined limit value for a total dose. In addition or alternatively, it is preferably determined whether an integral (over a period of time) or a sum of measured values of several measurements (corresponds to a dose) over a radiation pulse exceeds a predefined limit value for a pulse dose.

For example, the air kerma rate can be measured in real time, for example in the collimator or on the beam generator with the aid of a scintillator, for example, a scintillator ceramic such as, for example, UFC as well as a photodiode and an amplification circuit (for example, a transimpedance amplifier) and a digitization circuit. The measurements are preferably taken at a time interval of less than one millisecond (sub-millisecond sampling). This allows an air kerma rate limitation to be directly implemented.

By taking many measurements in a short time, the air kerma for each pulse of a serial recording or fluoroscopy series can be determined directly by integrating or adding up the air kerma rate over the time of acquisition and a reliable examination can be executed without long adjustment processes of the beam. The air kerma can also be calculated for individual X-ray pulses in a radiography application. This makes it possible to change from the indirect measurement of the air kerma used (via the indirect route of the kWs) to a direct measurement, which offers an advantage in terms of accuracy and, above all, also takes into account the ageing of the emitter.

By comparing the measured values over a long period of time (several examinations), embodiments of the present invention can also be used to detect ageing processes of the beam generation system and to compensate for them by making suitable adjustments to the control of the emitter, thus extending the service life of the X-ray beam.

Furthermore, using the measured values it is also possible to predict the ageing processes of the beam in the future and to make a prediction about the time of failure.

Likewise, embodiments of the present invention make it possible to detect flashovers (arcing) in the emitter as well as other disadvantageous dropouts in the X-ray radiation which can also be used to predict the service life or to indicate the beam quality.

In particular, embodiments of the present invention can be realized in the form of a processor unit with suitable software. The processor unit may, for example, have one or more cooperating microprocessors or the like for this purpose. In particular, it can be realized in the form of suitable software program parts in the processor unit. A largely software-based realization has the advantage that previously used processor units can also be easily retrofitted via a software or firmware update in order to operate in the manner according to embodiments of the present invention. In this respect, the object is also achieved by a corresponding computer program product with a computer program which can be loaded directly into a storage facility (also referred to as a storage device) of a processor unit, with program sections, in order to execute all the steps of the method according to embodiments of the present invention when the program is executed in the processor unit. Besides the computer program, such a computer program product may comprise additional components such as, for example, documentation and/or additional components, including hardware components such as, for example, hardware keys (dongles, etc.) for using the software.

A computer-readable medium, for example a memory stick, a hard disk or another transportable or permanently installed data carrier, on which the program sections of the computer program which can be read and executed by a processor unit are stored, can be used for transport to the processor unit and/or for storage on or in the processor unit.

Further, particularly advantageous embodiments and developments of embodiments of the present invention will emerge from the dependent claims and the following description, it also being possible for the claims of one claim category to be developed analogously to the claims and description parts of another claim category and in particular, it also being possible to combine individual features of different exemplary embodiments or variants to form new exemplary embodiments or variants.

A preferred regulating apparatus is designed to regulate the intensity of the radiation intensity emitted by the radiation source based on a measurement of the radiation sensor within a period of time (latency) shorter than 1 ms, preferably shorter than 0.8 ms, preferably shorter than 0.5 ms after this measurement. A beam must therefore be switched off after less than 0.8 ms, for example, if a limit value is exceeded. Preferably, the regulating apparatus is designed to perform the regulation in a pulsed emission of the beam when measuring a pulse before the emission of a fixed number of N pulses after the measurement, preferably where N<3. It is preferable that the beam is switched off before emission of the next radiation pulse once it has been determined that a limit value for a dose has been exceeded.

A preferred regulating apparatus is designed to store and evaluate a multiplicity of measurements of the intensity of the radiation from the radiation source by the radiation sensor at different times. Preferably, the measurements are taken at intervals shorter than 1 ms, particularly preferably shorter than 0.1 ms. In the case of pulsed radiation, it is preferable for a pulse to be measured at different times on several occasions. Alternatively or in addition, it is preferable that several pulses are measured in the case of pulsed radiation. Alternatively or in addition, it is preferable that measurements are performed during different examinations (to determine signs of ageing).

The control unit is preferably designed to control the intensity of the radiation emitted from the radiation source based on an integral, a sum and/or a chronological sequence of these measurements. Preferably, several measurements are taken within the defined period of time for the latency (for example, 2 ms) and regulation is based on these measurements.

A preferred regulating apparatus is designed to calculate a dose rate, in particular an air kerma rate, and/or to calculate a dose, in particular an air kerma, from values measured by the radiation sensor and a determined calibration function. The measured values are preferably converted and adjusted to dose values. The limit values can then be dose values directly, preferably a maximum total dose and/or a maximum pulse dose and/or a maximum dose rate.

It is preferable that the regulating apparatus is designed first to convert the values using the calibration function and then to integrate them over a predetermined period of time or first to integrate them and then to apply the calibration function to the integral.

A preferred regulating apparatus is designed to calculate a limit value for measured values based on a dose rate, in particular an air kerma rate, and/or a dose, in particular an air kerma, and an ascertained calibration function. The limit values are preferably converted and adapted to measured values. The limit values can then directly be maximum permissible measured values or maximum sums or integrals (over time) of measured values.

A preferred regulating apparatus is characterized in that the regulating apparatus is designed for the extrapolation of the intensity of the radiation from the radiation source and/or the dose at a later time based on the chronological sequence of the measurements. In this respect, for example, the increase of several measured values can be considered over a certain time and it can be determined when a limit value will be reached with the last calculated increase. However, ageing of the radiation source can also be determined by examining the increase.

The regulating apparatus is preferably designed to perform several measurements within the period of time. In this case, the regulating apparatus is preferably also designed for an examination and to determine an expected switch-off time of the radiation generator of the radiation source at a future time. In particular, the differences between successive measured values are used to determine when a limit value for a dose is reached.

Preferably, the regulating apparatus is designed to perform several measurements for different examinations. In this case, the regulating apparatus is preferably also designed to determine the ageing of the radiation generator of the radiation source at a future time. This can be deduced from a continuous decrease in beam intensity. However, flashovers (arcing) in the emitter and other dropouts in the X-rays can also be detected and these findings can also be used to predict the service life.

A preferred regulating apparatus is characterized in that the radiation sensor comprises or is an ionization chamber (for example, a semiconductor ionization chamber or an air ionization chamber). Alternatively or in addition, the radiation sensor comprises a photosensor, for example, a photomultiplier or a photodiode, and possibly a suitable amplification and digitization circuit, preferably together with a scintillator. Preferably, the radiation sensor comprises several photosensors, in particular with different prefilters. The radiation sensor preferably comprises a dose sensor or a direct conversion sensor.

A preferred regulating apparatus is designed for wireless or wired real-time capable communication between the radiation sensor and the control unit and/or through the regulating apparatus to components to be regulated. Transmission must take place within a defined period in order to maintain the defined latency. As some time is also required for measurement and comparison, communication should take place in less than 0.5 ms.

A preferred medical technology system is characterized in that the radiation sensor measures radiation in or on the radiation generator or in or on a collimator downstream of the radiation generator. The regulating apparatus can already be integrated into the collimator, be integrated at the output of an X-ray emitter or even into the X-ray emitter.

The radiation sensor preferably measures radiation at the edge of a radiation cone or scattered radiation. The regulating apparatus is then designed to carry out regulation based on the measured radiation.

Particularly preferably, the regulating apparatus is designed to switch off the beam of a radiation source when a measurement exceeds a predefined limit value for a dose rate, and/or an integral or a sum of values of several measurements exceeds a predefined limit value for a total dose, and/or an integral or a sum of values of several measurements over a radiation pulse exceeds a predefined limit value for a pulse dose.

The regulating apparatus is preferably designed to calculate a future time for a pulsed beam at which a predefined limit value for a dose or a dose rate is exceeded and to initiate a switch-off of the beam of the radiation source before the limit value is exceeded. The regulating apparatus is preferably designed to switch off the beam of the radiation source if the calculation shows that a predefined limit value is exceeded for a dose rate and/or for a total dose and/or for a pulse dose.

A preferred medical technology system comprises an image detector designed to detect the radiation emitted from the radiation source for an image recording, the image detector preferably being used as a radiation sensor of the regulating apparatus or for calibration of the radiation sensor. The regulating apparatus is preferably designed to analyze detector gray values of the image detector for control of the radiation emitted from the radiation source or for calibration of the radiation sensor, preferably based on a mean and/or median and/or another statistical measure of pixel values in a predetermined or dynamically defined area of the image detector.

A preferred method is based on pulsed beam operation of the radiation source. It comprises the following steps:
measurement of the intensity of the first N beam pulses of the radiation source, in particular in the case of a beam ramp-up, with preferably N<3, with the radiation sensor and the image detector and preferably also measurement of the current and/or the voltage of the radiation generator,
specification of a number of limit values corresponding to a maximum dose rate and/or a maximum total dose and/or a maximum pulse dose for the respective examination,
calibration of the regulating apparatus so that measurements of the radiation sensor can be compared with the number of limit values,
continuation of measurements of the intensities of further pulses with the radiation sensor,
ascertainment at least based on these measurements as to whether a limit value of the number of limit values has been exceeded, and if so: switch-off of the radiation.

For example, the intensity of a first beam pulse of the radiation source can be used when adjusting the radiation source. This pulse usually does not have the final intensity, but this can be compensated for by way of calculation. Measurements of the current and/or voltage of the radiation generator can improve calibration.

The limit values can, as mentioned above, correspond to a maximum dose rate and/or a maximum total dose and/or a maximum pulse dose, but they can also be converted in such a way that they can be directly compared with the measured values or a sum of measured values. The latter can save computing time.

The regulating apparatus can therefore be calibrated in such a way that the limit values are calibrated accordingly or that the measured values are calibrated. It should be noted that the calibration does not necessarily depend only on the system used but may also depend on the patient being examined. The limit values may also depend on the patient, which makes calibration very important.

For example, a first pulse with a low dose is delivered by an X-ray system. The detector value produced by an image detector (for example, a flat-panel detector) at this dose is evaluated and related to a target detector gray value. The pulse dose of this pulse (without a patient) is known from previous measurements. The ratio of the dose of this first pulse to the dose of the normal pulses is also known. The limit values can then be determined using the measured detector gray value, for example, depending on the absorption of the radiation by the patient or depending on the age of the patient (for example, lower limit values are set for children).

Assuming that the measured values of the radiation sensor of the regulating apparatus according to embodiments of the present invention are in a fixed relationship (for example, linear or according to a predefined function) with the detector gray value of the image detector, a calibration function can now be determined, which indicates, for example, how the measured values are related to a current dose rate.

In practice, a maximum total dose of the patient and a maximum dose rate as well as a maximum pulse dose might depend on general specifications. A power limit value and a pulse limit value can then be determined based on the specifications and an overall limit value can be determined with the measurements of the image detector. The overall limit values can then be converted so that they are comparable with the measured values and calibrated via the measurements of the image detector so that the control takes place when the beam exceeds a maximum dose rate or the maximum pulse dose or the maximum total dose have been reached.

The measurements of the intensities are of course taken over the entire examination so that it can be ascertained when a limit value has been exceeded or is at risk of being exceeded. If a limit value has been exceeded or an evaluation shows that it would be exceeded with the next pulse, the radiation is switched off.

Preferably, the calibration is carried out using a fixed formula. For example, it can be specified that the dose rate for a subsequent pulse is x-times the N radiation pulses. A fixed function can also be used to appropriately scale measured values depending on a measured current or voltage of the radiation source.

Preferably, the calibration is carried out iteratively with the measurement of several pulses. As the image detector operates during image acquisition, its information can also be used continuously for calibration.

Preferably, the calibration is calculated based on measured radiation flux and/or radiation energy. A fixed function can be used here as a function of the corresponding variables, in particular together with information from the image detector. In particular, the image detector can provide information regarding radiation flux and/or radiation energy.

The use of AI-based methods (AI: “Artificial Intelligence”) for the method according to embodiments of the present invention is preferable. Artificial intelligence is based on the principle of machine-based learning and is generally performed using an algorithm which is capable of learning and has been trained accordingly. The term “machine learning” is often used for machine-based learning, the principle of “deep learning” also being included in this case. An appropriately trained machine-learning model is particularly advantageous for making predictions about the risk of exceeding a limit value. Even if training takes a comparatively long time, results can be delivered very quickly with trained models (within the corresponding control time TR).

### BRIEF DESCRIPTION OF THE DRAWINGS

The present invention is explained in more detail hereinafter with reference to the attached figures using exemplary embodiments. The same components are provided with identical reference characters in the various figures. The figures are generally not to scale. The figures show:

FIG. 1A rough diagrammatic representation of a radiography system,

FIG. 2A radiation source with a regulating apparatus according to embodiments of the present invention,

FIG. 3A block diagram of the method sequence,

FIG. 4A diagram regarding the process and the effect of the method,

FIG. 5A diagram regarding the control of radiation pulses.

### DETAILED DESCRIPTION

The following explanations are based on the assumption that the radiography system 1 is a conventional X-ray device. In principle, however, the method can also be used on other medical technology systems 1 which use radiation R to record images or treat patients P.

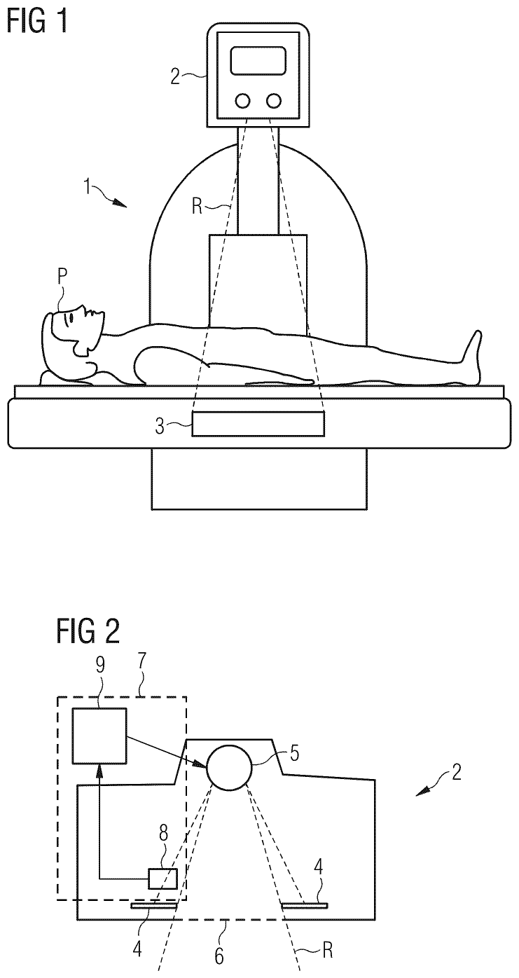

FIG. 1 shows a rough diagrammatic view of a radiography system 1 as an example of a medical technology system 1 comprising an X-ray source 2 as an example of a radiation source 2 and an image detector 3. During radiography, an X-ray beam R shines through a patient P so that the radiation R impinges on the opposite image detector 3.

FIG. 2 shows a radiation source 2 with a regulating apparatus 7 according to embodiments of the present invention. The radiation source comprises a radiation generator 5 (here an X-ray tube) and a collimator. Arranged at the top is the radiation generator 5 which generates an X-ray beam R during operation which emerges from the top of the exit window 6. The range of the X-ray beam R is limited by collimator plates 4, which are usually adjustable.

The regulating apparatus 7 comprises a radiation sensor 8, which in this example is arranged above the collimator plates 4 and measures the intensity D of radiation R from the radiation source 2 in a part of the X-ray cone which is blanked out for the recording. From the measured intensity, at least after calibration, an overall intensity of the X-ray beam R can be extrapolated.

The regulating apparatus 7 also comprises a control unit 9 designed to control the intensity D of the radiation R emitted by the radiation source 2. The radiation sensor 8 measures the radiation R from the radiation source 2 and passes the measured values on to the control unit 9 (bottom arrow), which in this case controls the radiation generator 5 directly in a defined period of time shorter than 2 ms after a measurement (right arrow).

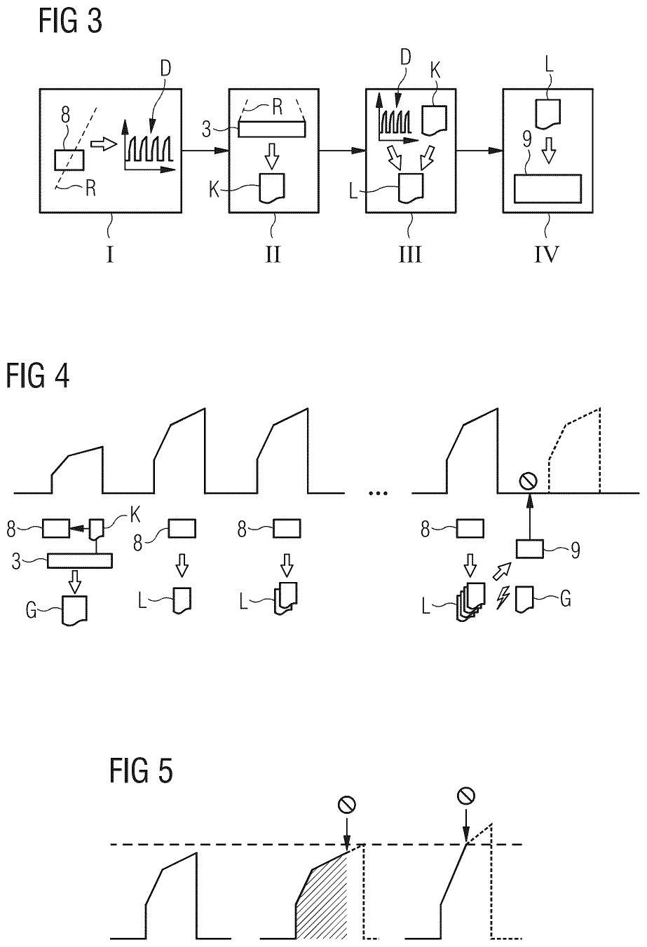

FIG. 3 shows a block diagram of the sequence of the method for dose limitation of a radiation source 2 of a medical technology system 1 as shown, for example, in FIG. 1.

In step I, the intensity D of the radiation R of the radiation source 2 is measured with the radiation sensor 8 of the regulating apparatus 7 (cf. FIG. 2). Measurements are taken for different radiation pulses using the radiation sensor 8. The measured values can be plotted as shown in the diagram.

In step II, images of the image detector 3 are analyzed, in particular the detector gray value and a calibration function K is calculated from this for the measured values or for limit values. In the example shown here, values for the air kerma rate are to be ascertained from the measured values.

In step III, the measured values together with the calibration function are converted into values for an air kerma rate. An air kerma value L is then determined, which can comprise an air kerma rate and/or an air kerma.

In step VI, the intensity of the radiation R emitted by the radiation source 2 is controlled by the control unit 9 of the regulating apparatus 7, at least when a limit value has been exceeded.

Exactly how this works is shown in the following figures.

FIG. 4 shows a diagram of the sequence and the effect of the method for dose limitation of a radiation source 2 of a medical technology system 1. In this example, pulsating radiation R is used, which is recorded here as pulses.

On the far left, the emission of the radiation begins with a reduced pulse. Using the image detector (and its detector gray value), a calibration function K is ascertained from this pulse which can be used to calibrate the measured values of the radiation sensor 8. As the behavior of the radiation at higher intensities is known, this calibration function can be scaled to higher intensities here. In cases where this is not possible, the calibration can be carried out using the normal pulses or the normal pulses can also be used for calibration.

The radiation sensor 8 now measures the intensities of the normal pulses during an image acquisition or therapy and an air kerma value L is calculated, for example, a sum of several air kerma values over one pulse.

This air kerma value L is measured for each pulse and added up to an air kerma (in this case, a total value).

After a number of pulses, it is determined that a limit value G for a total dose has now been exceeded (indicated by the flash). The control unit now switches off the radiation source so that no further pulses are emitted (the dotted pulse is intended to indicate that further pulses are missing).

FIG. 5 shows a sketch for controlling radiation pulses. Further control options are indicated here. While FIG. 4 showed the example of the behavior in the event of a maximum total dose, the possibilities of interrupting the radiation R when a maximum pulse dose (hatched area in the center) or a maximum dose rate (exceeding the dashed line on the right) is exceeded are shown here.

If many measurements are taken within one pulse, it can be determined that a maximum pulse dose has been exceeded and the radiation source 2 can be switched off for this pulse (center). It can then also be determined that a maximum dose rate has been exceeded and the radiation source 2 for this pulse can likewise be switched off (on the right).

Finally, it is pointed out once again that the present invention described in detail above merely concerns exemplary embodiments which can be modified in many ways by a person skilled in the art without departing from the scope of the present invention. Moreover, the use of the indefinite article “a” or “an” does not exclude the possibility that the relevant features may also be present more than once. Likewise, terms such as “unit” do not exclude the possibility that the relevant components consist of several interacting sub-components which may also be spread over a wide area. The term “a number” is to be read as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between

elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SOC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processor) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description.

However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A regulating apparatus for dose limitation of a radiation source configured to generate radiation via a radiation generator, the regulating apparatus comprising:
a radiation sensor configured to measure an intensity of radiation emitted from the radiation source;
a control unit configured to regulate an intensity of the radiation emitted from the radiation source, wherein
the regulating apparatus is configured to regulate the radiation emitted from the radiation source based on a measurement by the radiation sensor within a period of time less than 2 ms after the measurement.

2. The regulating apparatus as claimed in claim 1, wherein the regulating apparatus is configured to regulate the intensity of the radiation emitted from the radiation source based on the measurement by the radiation sensor within a period of time less than 1 ms after the measurement.

3. The regulating apparatus as claimed in claim 1, wherein
the regulating apparatus is configured to store and evaluate a plurality of measurements of the intensity of the radiation emitted from the radiation source by the radiation sensor at different times, and
at least one of
in case of pulsed radiation, a pulse is measured at different times on several occasions,
in the case of pulsed radiation, several pulses are measured, or
measurements are carried out for different examinations.

4. The regulating apparatus as claimed in claim 3, wherein the regulating apparatus is configured to
calculate at least one of a dose rate or a dose based on a calibration function and values measured by the radiation sensor and, or
calculate a limit value for measured values based on the calibration function and at least one of the dose rate or the dose.

5. The regulating apparatus as claimed in claim 3, wherein the regulating apparatus is configured to
extrapolate at least one of the intensity of the radiation from the radiation source or a dose at a later time based on a chronological sequence of measurements.

6. The regulating apparatus as claimed in claim 1, wherein the radiation sensor at least one of (i) includes or is an ionization chamber or (ii) includes a photosensor.

7. The regulating apparatus as claimed in claim 1, wherein the regulating apparatus is configured for wireless or wired real-time capable communication at least one of (i) between the radiation sensor and the control unit or (ii) through the regulating apparatus to components to be regulated.

8. A medical technology system for at least one of imaging or radiotherapy, the medical technology system comprising:
a radiation source with a radiation generator; and
a regulating apparatus as claimed in claim 1, wherein the regulating apparatus is configured to
regulate the intensity of the radiation emitted from the radiation source, and
initiate, based on the measurement by the radiation sensor, a reduction in beam intensity or switch-off of a beam of the radiation source in response to the measurement being outside a threshold range of values.

9. The medical technology system as claimed in claim 8, wherein
the radiation sensor is configured to measure radiation in or on the radiation generator or in or on a collimator downstream of the radiation generator.

10. The medical technology system as claimed in claim 8, wherein the regulating apparatus is configured to
calculate a future time for a pulsed beam at which a threshold limit value for a dose or a dose rate is exceeded, and
initiate the switch-off of the beam of the radiation source before the threshold limit value is exceeded.

11. The medical technology system as claimed in claim 8, further comprising:
an image detector configured to detect the radiation emitted from the radiation source for an image recording, wherein
the image detector is used as the radiation sensor of the regulating apparatus or to calibrate the radiation sensor,
the regulating apparatus is configured to evaluate detector gray values of the image detector for regulating the radiation emitted from the radiation source or for calibrating the radiation sensor, based on at least one of a mean, a median or another statistical measure of pixel values in a defined range of the image detector.

12. A method for dose limitation of a radiation source of the medical technology system as claimed in claim 8, the method comprising:
measuring an intensity of the radiation emitted from the radiation source with the radiation sensor of the regulating apparatus of the medical technology system;
comparing a number of threshold limit values with at least one measured intensity or an integral or a sum of the at least one measured intensity;
in response to a threshold limit value being exceeded regulating, by the control unit, the intensity of the radiation emitted from the radiation source.

13. The method as claimed in claim 12, wherein
the method is for pulsed beam operation of the radiation source, and
the method further includes
measuring an intensity of a first N beam pulses of the radiation source with the radiation sensor and an image detector,
determining a number of limit values corresponding to at least one of a maximum dose rate, a maximum total dose or a maximum pulse dose for a respective examination,
calibrating the regulating apparatus such that measurements of the radiation sensor are comparable with the number of limit values,
continuing to measure intensities of further pulses with the radiation sensor,
ascertaining, at least based on the measured intensities, whether a limit value of the number of limit values has been exceeded, and
switching off the radiation in response to ascertaining that the limit value has been exceeded.

14. A non-transitory computer program product comprising commands that, when executed by a computer, cause the computer to perform the method as claimed in claim 12.

15. A non-transitory computer-readable storage medium storing computer-executable instructions that, when executed by a computer, cause the computer to perform the method as claimed in claim 12.

16. The regulating apparatus as claimed in claim 1, wherein the regulating apparatus is configured to regulate the radiation in a pulsed emission of a beam when measuring a pulse before emission of a fixed number of N pulses after the measurement.

17. The regulating apparatus of claim 3, wherein the control unit is configured to regulate the intensity of the radiation emitted from the radiation source based on at least one of an integral sequence of the plurality of measurements or a chronological sequence of the plurality of measurements.

18. The regulating apparatus of claim 4, wherein the dose rate is an air kerma rate and the dose is an air kerma.

19. The regulating apparatus of claim 4, wherein the regulating apparatus is configured to
convert the measured values using the calibration function and integrate the converted values over a period of time, or
integrate the measured values and apply the calibration function to the integrated values.

20. The regulating apparatus of claim 5, wherein the regulating apparatus is configured to at least one of
perform several measurements within a period of time of an examination and determine an expected switch-off time, or
perform several measurements for different examinations and determine aging of the radiation generator of the radiation source at a future time.

21. The regulating apparatus of claim 6, wherein the radiation sensor includes a photosensor and a scintillator.

22. The regulating apparatus of claim 6, wherein the radiation sensor includes a plurality of photosensors with different prefilters.

23. The medical technology system of claim 8, wherein
the radiation source is configured for pulsed beam operation, and
the regulating apparatus is configured to regulate the radiation source such that on reaching a maximum dose, the radiation source is switched off before transmission of a next pulse.

24. The medical technology system of claim 9, wherein
the radiation sensor is configured to measure radiation at an edge of a radiation cone or scattered radiation, and
the regulating apparatus is configured to
regulate the radiation based on the measured radiation, and
switch off the beam of the radiation source in response to at least one of
a measurement exceeding a threshold limit value for a dose rate,
an integral or a sum of values of several measurements exceeding a threshold limit value for a total dose, or
an integral or a sum of values of several measurements over a radiation pulse exceeding a threshold limit value for a pulse dose.

25. The medical technology system as claimed in claim 10, wherein the regulating apparatus is configured to
switch off the beam of the radiation source in response to the calculation indicating that the threshold limit value for at least one of the dose rate, a total dose or a pulse dose is exceeded.

26. The method of claim 12, wherein at least one of
the measuring is performed via the radiation sensor for different radiation pulses,
signals from the control unit are transmitted directly to the radiation source, or
the method further includes ascertaining at least one of
whether a measurement exceeds a threshold limit value for a dose rate,
whether an integral or a sum of measured values of several measurements exceeds a threshold limit value for a total dose, or
whether an integral or a sum of measured values of several measurements over a radiation pulse exceeds a threshold limit value for a pulse dose.

27. The method as claimed in claim 13, wherein at least one of N<3,
the method further includes measuring at least one of a current or a voltage of the radiation generator, or
the calibrating is at least one of
performed with a fixed formula,
performed iteratively with a measurement of several pulses, or
based on at least one of measured radiation flux or radiation energy.
